(19) United States
(12) Patent Application Publication
(10) Pub. No.: US 2006/0264904 A1
(43) Pub. Date: Nov. 23, 2006

(54) Medical device

(76) Inventors: Walter Lynn Kerby, South Jordan, UT (US); William Webb, Riverton, UT (US)

Correspondence Address: CROMPTON, SEAGER & TUFTE, LLC, 1221 NICOLLET AVENUE, SUITE 800, MINNEAPOLIS, MN, 55403-2420, US

(21) Appl. No.: 11/125,987

(22) Filed: May 9, 2005

(51) Int. Cl. A61M 25/00

(52) U.S. Cl. 604/523

(57) ABSTRACT

A medical device including an alternative shaft configuration, structure, assembly, and method for making. For example, some shaft configurations relate to the use of an inner and an outer tubular member. Additionally, some alternative distal tip structures, assemblies, and methods for elongated medical devices are disclosed. Additionally, some alternative structures, assemblies, and methods for proximal shaft constructions and for connecting manifolds to medical devices are disclosed. For example, the shaft of a medical device may be provided with a flared proximal portion that is adapted to mate with structure on a manifold assembly. Additionally, the manifold assembly may include structures, for example, protrusions and/or recessed portions, which are adapted to mate with the flared section of the shaft.

### FIELD OF THE INVENTION

The invention relates generally to medical devices. More specifically, the invention relates to a medical device, such as a catheter or the like, including an elongated shaft defining a lumen.

### BACKGROUND

Elongated medical devices are commonly used to facilitate navigation through and/or treatment within the anatomy of a patient. A variety of elongate medical devices for intracorporal use, such as catheters, endoscopes and the like, have been developed over the past several decades. Because the anatomy of a patient may be very tortuous, it is often desirable to combine a number of performance features in such devices. For example, it is sometimes desirable that the device have a relatively high level of pushability and torqueability, particularly near its proximal end. It is also sometimes desirable that a device be relatively flexible, particularly near its distal end. A number of different elongated medical device structures and assemblies are known, each having certain advantages and disadvantages. However, there is an ongoing need to provide alternative elongated medical device structures, assemblies, and methods.

For example, a number of different shaft structures, assemblies, and methods for medical devices are known, but there is an ongoing need to provide alternatives to those that are known. Furthermore, a number of different distal tip structures, assemblies, and methods are known for medical devices, but again, there is an ongoing need to provide alternatives. Additionally, it is generally known to provide a manifold and/or hub and strain relief assembly on a medical device, such as on a catheter, or the like. However, there is an ongoing need to provide alternative structures, assemblies, and methods for connecting manifolds to medical devices.

### SUMMARY OF SOME EMBODIMENTS

The invention provides design, material, and manufacturing method alternatives for medical devices, such as catheters and the like. Some embodiments may relate to providing alternative shaft structures, assemblies, and methods for elongated medical devices, such as catheters. Other embodiments may relate to alternative distal tip structures, assemblies, and methods for elongated medical devices. Some additional embodiments may relate to alternative structures, assemblies, and methods for proximal shaft constructions and connecting manifolds to medical devices.

The above summary of some embodiments is not intended to describe each disclosed embodiment or every implementation of the present invention. The Figures, and Detailed Description which follow more particularly exemplify these embodiments.

### BRIEF DESCRIPTION OF THE DRAWINGS

The invention may be more completely understood in consideration of the following detailed description of various embodiments of the invention in connection with the accompanying drawings, in which:

FIG. 1 is a partial side plan view of a medical device in accordance with one example embodiment of the invention, shown as a catheter, for example a delivery, guide or diagnostic catheter;

FIG. 2 a partial cross sectional view of a portion of the medical device of FIG. 1;

FIG. 3 is a partial cross sectional view of a portion of the shaft of the medical device of FIG. 1, including one example of a distal tip configuration;

FIG. 4 is a cross sectional view of a proximal portion of the medical device of FIG. 1, showing the manifold assembly and a part of the proximal portion of the shaft;

FIG. 4A is a magnified view of the area defined in circle 4a of FIG. 4;

FIG. 5 is an exploded cross-sectional view of a proximal portion of the medical device of FIG. 1, showing the manifold assembly, including the hub portion and the strain relief portion, and a part of the proximal portion of the shaft;

FIG. 6 is a cross sectional view of a proximal part of an inner tubular assembly of a shaft construction prior to attachment of the outer tubular member and prior to flaring of the proximal end;

FIG. 7 is a cross sectional view of the part of an inner tubular assembly of FIG. 6, showing attachment of the outer tubular member and the insertion of a mandrel to flare the proximal end;

FIG. 8 is a cross sectional view of the part of an inner tubular assembly of FIG. 7, showing attachment of a first layer of material about the proximal end;

FIG. 9 is a cross sectional view of the part of an inner tubular assembly of FIG. 8, showing attachment of a second layer of material about the proximal end; and

FIG. 10 is a cross sectional view of an alternative proximal part of tubular shaft including a flared proximal end.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION OF SOME EMBODIMENTS OF THE INVENTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following detailed description should be read with reference to the drawings in which similar elements in different drawings are numbered the same. The drawings, which are not necessarily to scale, depict illustrative embodiments and are not intended to limit the scope of the invention.

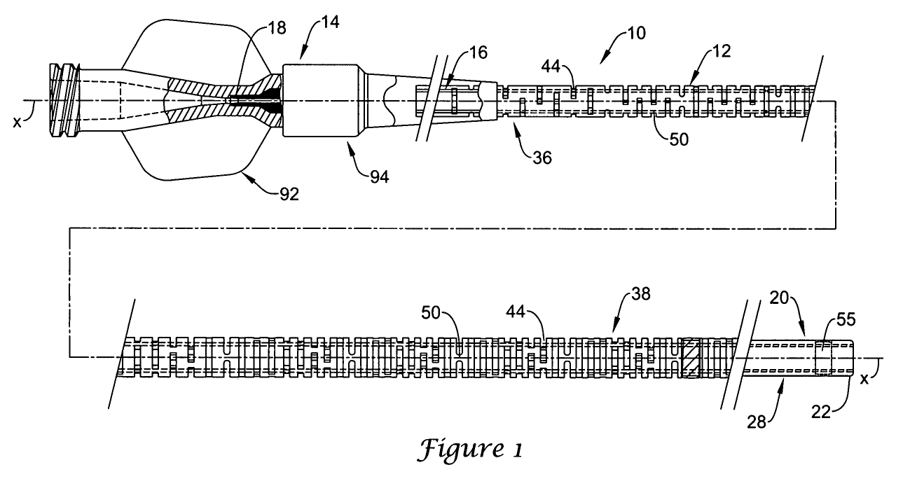

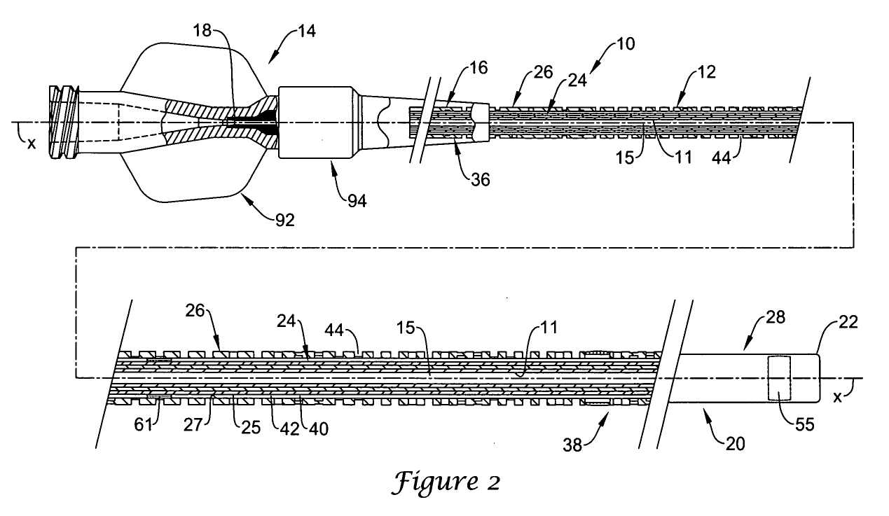

Refer now to FIGS. 1 and 2, which illustrates a medical device 10 in accordance with one example embodiment. In general, the medical device may be a catheter 10, and can include a generally elongate shaft 12 extending along a central or longitudinal axis x. The axis x extends along the length of the catheter 10 and necessarily follows the shape and/or curvature of the shaft 12. The shaft 12 can include a proximal portion 16 having a proximal end 18, and a distal portion 20 having a distal end 22. A distal tip 28 may be disposed at the distal portion 20, and a manifold assembly 14 may be connected at the proximal portion 16.

As an initial matter, it should be appreciated that while the medical device 10 is depicted as an intravascular catheter 10, and in particular, an intravascular delivery, guide and/or diagnostic catheter 10, this is for the purposes of illustration only. Other medical devices embodying aspects of the invention may relate to virtually any medical device including an elongate shaft defining an elongated lumen, and optionally including a manifold. For example, other embodiments may relate to medical devices such as a balloon catheter, an atherectomy catheter, a drug delivery catheter, a stent delivery catheter, an endoscope, an introducer sheath, a fluid delivery device, other infusion or aspiration devices, device delivery (i.e. implantation) devices, and the like. Thus, while the Figures and descriptions below are directed toward a delivery, guide, and/or diagnostic catheter, in other applications the structure and/or sizes in terms of diameter and length may vary widely, depending upon the desired properties of a particular device.

Additionally, it should be appreciated that the shaft 12, manifold assembly 14, and distal tip 28 can generally include any of a broad variety of structures and/or configurations. It should be understood that the particular configurations and structures shown and described herein are by way of example only, and that a broad variety of alternative structures and/or configurations may be used without departing from the spirit and scope of the invention as claimed.

With this understanding, what follows is a discussion of some example embodiments of catheters 10 including such structures. First, a discussion of example embodiments of shaft constructions 12 will be provided, with a discussion of at least some examples of distal tip 28 constructions. Thereafter, a discussion related to example embodiments of shaft 12 and manifold assembly 14 configurations and methods used to attach the manifold assembly 12 to the proximal portion 16 of the shaft 12 will be provided.

The shaft 12 can be manufactured, include structure, and be made of materials so as to provide the desired characteristics of the catheter 10, depending upon the intended use. For example, the shaft 12 can be provided and/or manufactured so as to maintain a desired level of flexibility, torquability and/or other characteristics appropriate for maneuvering the catheter 10 as desired, for example, through the vasculature of a patient. As such, it should be understood that there is a broad range of possible shaft constructions that may be used—including those particularly discussed herein and others. Some other examples of suitable catheter shaft constructions and materials can be found in U.S. Pat. Nos. 5,569,218; 5,603,705; 5,674,208; 5,680,873; 5,733,248; 5,853,400; 5,860,963; 5,911,715; and 6,866,665, all of which are incorporated herein by reference. Some additional examples of shaft constructions include those disclosed in U.S. patent application Ser. No. 10/238,227 (Publication No. US-2004-0045645-A1), which is also incorporated herein by reference.

The shaft 12 may have a length and an outside diameter appropriate for its desired use, for example, to enable intravascular insertion and navigation. For example, in some embodiments, the shaft 12 may have a length in the range of about 1-300 centimeters or more, or in some embodiments in the range of about 20 cm-250 cm, and an outside diameter in the range of about 1 F to about 20 F, or in some embodiments, in the range of about 1 F to about 10 F. Additionally, although depicted as including a generally round outer diameter and a round cross-sectional shape, it can be appreciated that the shaft 12 can include other outer diameter and/or cross-sectional shapes or combinations of shapes without departing from the spirit of the invention. For example, the outer diameter and/or cross-sectional shape of the generally tubular shaft 12 may be oval, rectangular, square, triangular, polygonal, and the like, or combinations thereof, or any other suitable shape, depending upon the desired characteristics.

In some embodiments, the catheter 10 can be a microcatheter including a shaft 12 that is adapted and/or configured for use within small anatomies of the patient. For example, some embodiments are particularly useful in treating targets located in tortuous and/or narrow vessels. Some examples of such vessels may include those in the neurovascular system, or in certain sites within the coronary vascular system, or in sites within the peripheral vascular system such as superficial femoral, popliteal, or renal arteries. The target site in some embodiments is a neurovascular site, such as a site in the brain, which is accessible only via a tortuous vascular path, for example, a vascular path containing a plurality of bends or turns which may be greater than about 90° turns, and/or involving vessels which are in the range of about 8 mm or less, and in some cases as small as 2-3 mm or less, in diameter. As such, in some embodiments, the shaft 12 can include an outside diameter in the range of approximately 1 F-4 F.

However, in other embodiments, the catheter 10 may be used in other target sites within the anatomy of a patient, in which case the shaft 12 would be so adapted. For example, the catheter 10 may be suited for other uses in the digestive system, soft tissues, or any other use including insertion into an organism for medical uses, and the shaft 12 could be appropriately adapted for such uses. For example, in some embodiments, the catheter 10 may be used as an introducer sheath, in which case the shaft 12 may be significantly shorter. The catheter 10 may also include additional structure and materials adapted for a particular use and/or procedure. For example, in some other embodiments, the shaft 12 may include additional devices or structures such as inflation or anchoring members, device deployment members, sensors, optical elements, ablation devices, or the like, or any of a broad variety of other structures, depending upon the desired function and characteristics of the catheter 10.

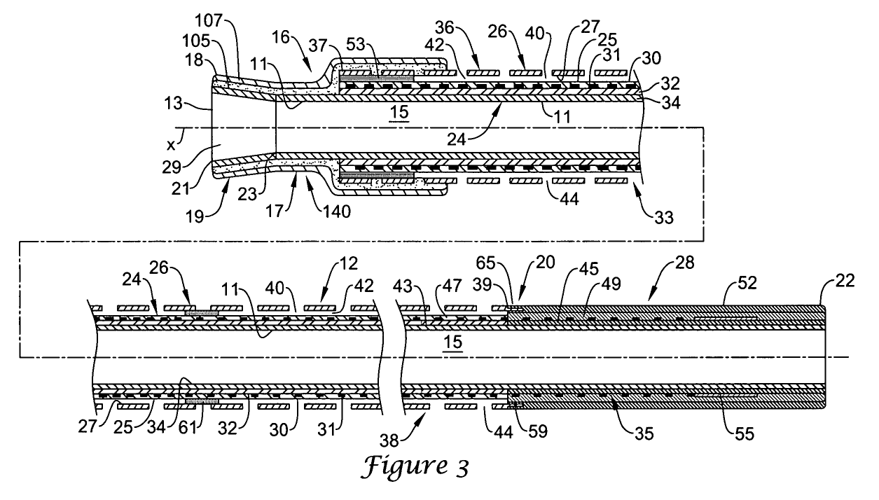

Referring now to FIG. 2, in at least some embodiments, the shaft 12 can have a generally tubular construction that includes at least one lumen 15 extending the length of the shaft 12 along the longitudinal axis x. This can also be seen with reference to FIG. 3, which is a partial cross-sectional view of the shaft 12 without the manifold assembly 14. The lumen 15 can be defined by an inner surface 11 of the shaft 12, and can have an inner diameter capable of transmitting fluids, or in some cases, receiving another medical device, such as a guidewire, a stent, a coil (such as an embolic coil, or the like), treatment particles (such as embolic particles, or the like), an ablation device, or another catheter, for example, a diagnostic catheter, a balloon catheter, a stent delivery catheter, or the like, or others. In some embodiments, the lumen 15 can be adapted and/or configured to accommodate another medical device having an outer diameter in the range of about 1 F to about 10 F.

In this embodiment, the shaft 12 includes a generally tubular construction including an inner tubular assembly and/or member 24, and an outer tubular assembly and/or member 26 disposed about at least a portion of the inner tubular member 24—however it should be understood that this is by way of example only. The inner tubular member 24 at least partially defines the inner surface 11 of the shaft 12, and thus defines the lumen 15.

The inner tubular member 24 can extend from a point within the distal portion 20 to a point within the proximal portion 16 of the shaft 12. The length of the inner tubular member 24 can vary, depending upon, for example, the length of the shaft 12, the desired characteristics and functions of the inner tubular member 24, and other such parameters. In some embodiments, the inner tubular member 24 can extend substantially the entire length of the shaft 12, for example, from a point adjacent the proximal end 18 to a point adjacent the distal end 22. For example, the length of the inner tubular member 24 can be in the range of about 1-300 centimeters or more, or in some embodiments in the range of about 20 cm-250 cm.

Referring to FIG. 3, the inner tubular member 24 can include a proximal portion 33 and a distal portion 35. The proximal and distal portions 33/35 can be any proximal or distal sections of the inner tubular member 24—however, in some cases the portions 33/35 can be defined with regard to the relative position of the inner and outer tubular members 24/26. For example, the distal portion 35 can be any portion of the inner tubular member 24 that extends distally beyond the distal end 39 of the outer tubular member 26, while the proximal portion 35 can be any portion of the inner tubular member 24 that is disposed within, or is proximal of a distal end 39 of the outer tubular member 26. In some embodiments, the distal portion 35 can have a length in the range of 0.5 cm or greater, or in the range of about 1 cm or greater, or 2 cm or greater, and in some embodiments in the range of about 3 to about 20 cm. In some embodiments, the distal portion 35 can be disposed within, and/or be apart of, or otherwise include a distal tip 28 construction, some examples of which will be discussed in more detail below.

The inner tubular member 24 may have an inner diameter, for example, defining the lumen 15, that is in the range of about 0.01 to about 0.05 inch in size, or in the range of about 0.015 to about 0.03 inch in size, or in the range of about 0.016 to about 0.026 inch in size. As indicated above, however, the lumen 15 (defined by the inner diameter of the inner tubular member 24) can be adapted and/or configured (e.g. sized) to accept other material, fluids, or medical devices, therein, and as such, the size of the lumen 15 can vary, depending upon the desired characteristics and intended use.

Additionally, the inner tubular member 24 can have an outer diameter that is in the range of about 0.011 to about 0.055 inch in size, or in the range of about 0.015 to about 0.03 inch in size, or in the range of about 0.019 to about 0.029 inch in size. It should be understood however, that these dimensions are provided by way of example embodiments only, and that in other embodiments, the size of the inner and outer diameter of the inner tubular member 24 can vary greatly from the dimensions given, depending upon the desired characteristics and function of the device.

The inner tubular member 24, or other portions of the shaft 12, may define one or more additional lumens depending upon the desired characteristics and function of the catheter 10, and such additional lumens can be shaped, size, adapted and/or configured the same as or different from lumen 15, depending upon the desired characteristic and functions.

The inner tubular member 24 may include and/or be made of any of a broad variety of materials and/or structures. The inner tubular member 24 may have a single-layer tubular construction or a multi-layer tubular construction, or a combination thereof. For example, the inner tubular member 24 may be a single tubular member formed by a single layer of material, or in other embodiment, may be formed by a plurality of tubular members and/or a plurality of layers of material that may be the same and/or different, but in combination form the inner tubular member 24. In yet other embodiments, some portions of the inner tubular member 24 can include a single layer construction, while other portions may include a multi-layer construction. Some examples of suitable materials can include, but are not limited to, polymers, metals, metal alloys, or composites or combinations thereof.

Some examples of some suitable polymers can include, but are not limited to, polyoxymethylene (POM), polybutylene terephthalate (PBT), polyether block ester, polyether block amide (PEBA), fluorinated ethylene propylene (FEP), polyethylene (PE), polypropylene (PP), polyvinylchloride (PVC), polyurethane, polytetrafluoroethylene (PTFE), polyether-ether ketone (PEEK), polyimide, polyamide, polyphenylene sulfide (PPS), polyphenylene oxide (PPO), polysufone, nylon, perfluoro(propyl vinyl ether) (PFA), polyether-ester, some adhesive resin, such as modified polyolefin resin, polymer/metal composites, etc., or mixtures, blends or combinations thereof, and may also include or be made up of a lubricous polymer. Some other potentially suitable polymer materials may include those listed below with reference to the outer tubular member 26. One example of a suitable polyether block ester is available under the trade name ARNITEL, and one suitable example of a polyether block amide (PEBA) is available under the trade name PEBAX®, from ATOMCHEM POLYMERS, Birdsboro, Pa. In some embodiments, adhesive resins may be used, for example, as tie layers and/or as the material of the structures. One example of a suitable adhesive resin is a modified polyolefin resin available under the trade name ADMER®, from Mitsui Chemicals America, Inc. Additionally, polymer material can in some instances be blended with a liquid crystal polymer (LCP). For example, in some embodiments, the mixture can contain up to about 5% LCP. This has been found in some embodiments to enhance torqueability.

Some examples of suitable metals and metal alloys can include stainless steel, such as 304V, 304L, and 316L stainless steel; nickel-titanium alloy such as a superelastic (i.e. pseudoelastic) or linear elastic nitinol; nickel-chromium alloy; nickel-chromium-iron alloy; cobalt alloy; tungsten or tungsten alloys; tantalum or tantalum alloys, gold or gold alloys, MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); or the like; or other suitable metals, or combinations or alloys thereof. In some embodiments, it is desirable to use metals, or metal alloys that are suitable for metal joining techniques such as welding, soldering, brazing, crimping, friction fitting, adhesive bonding, etc.

Referring to FIG. 3, at least a portion of the inner tubular member 24 can have a multi-layer tubular construction. The example shown includes an inner layer 34, an intermediate layer 32 disposed about the inner layer 34, a reinforcing layer 31 disposed about the intermediate layer 32, and an outer layer 30 disposed about the reinforcing layer 31 and the intermediate layer 32. It should be understood that more or fewer layers can be used, with or without one or more reinforcing layers, depending upon the desired characteristics of the inner tubular member 24. Additionally, in other embodiments, the layers could be arranged differently to achieve desired properties. For example, the reinforcing layer 31 could be disposed at a different radial location, could be disposed entirely within another layer, could be disposed on the outer surface of the inner tubular member 24, or, as indicated above, could simply be absent. Furthermore, while the layers 30, 32 and 34 are described, these layers may be provided separately but form a single and/or unitary layer and/or structure. Some or all of the plurality of layers, for example layers 30, 31, 32, 34, may be made of any suitable material, for example, those discussed above for use in the inner tubular member 24.

In some embodiments, the inner layer 34 may include a lubricious polymer such as HDPE or PTFE, for example, or a copolymer of tetrafluoroethylene with perfluoroalkyl vinyl ether (PFA) (more specifically, perfluoropropyl vinyl ether or perfluoromethyl vinyl ether), or the like. In some particular embodiments, a PTFE tube is used as the inner layer 34, which can extend the length of the inner tubular member 24.

Furthermore, in some embodiments, the intermediate and outer layers 32/30 may each individually include a flexible polymer, for example a polymer material having a durometer in the range of about 5 D to about 90 D. For example, the intermediate and/or outer layers 32/30 can include or be made up of one or more tubular segments of a PEBA, a polyether-ester elastomer, or other like material. The durometer of the material used to form the intermediate and/or outer layers 32/30 may be the same, or may vary from one another—depending upon the characteristics desired. For example, the intermediate layer 32 may be made of a material having a higher durometer than the material of the outer 30 layer along at least a portion of the inner tubular member 24. In other embodiments, the reverse may be true, and in yet other embodiments, the two layers 30/32 may include the material having the same or similar flexibility characteristics.

In some embodiments, one or both of the layers 30/32 can be made up of a plurality of tubular segments including materials having different flexibility characteristics to impart varying degrees of flexibility to different longitudinal sections of the intermediate and/or outer layers 32/30. For example, in some embodiments, one or both of the layers 30/32 can include one or more proximal segments (e.g. 43/47) and one or more distal segments (e.g. 45/45). In some cases, the one or more proximal segments (e.g. 43/47) in either one or both layers 30/32 may include material having a higher durometer than the material included in the distal segment (e.g. 45/45) of each or both respective layer 30/32. Such a construction may be used, for example, to render a more distal portion of the inner tubular member 24 more flexible. Such an arrangement can also be helpful, for example, in providing a flexible distal tip construction, or a portion thereof.

For example, referring to the embodiment shown in FIG. 3, the intermediate layer 32 may include a proximal portion 43 including and/or made of a flexible polymer, such as a PEBA, a polyether-ester elastomer, or other like material, having a durometer in the range of about 40 D to about 70 D. The intermediate layer 32 may also include a distal portion 45 including and/or made of a flexible polymer having a durometer in the range of about 15 D to about 35 D. Additionally, the outer layer 30 may include a proximal portion 47 including and/or made of such a flexible polymer having a durometer in the range of about 25 D to about 55 D. The outer layer 30 may also include a distal portion 49 including and/or made of such a flexible polymer having a durometer in the range of about 15 D to about 35 D.

The one or more reinforcing layer 31, if present, can be constructed with any suitable materials and structures to impart the desired characteristics to the inner tubular member 24. The reinforcing layer 31 can include one or more support members that can comprise, for example, a braid, a coil, a filament or wire, or series of such structures, or the like, including material and/or structure adapted to provide the desired characteristics. Examples of suitable materials for constructing the reinforcing layer include polymers, metals, or metal alloys such as those discussed above, or the like, or any of a broad variety of other suitable material.

In some embodiments, the reinforcing layer 31 can be a coil 31. The coil 31 may be formed of an elongated filament (e.g. wire, ribbon, or the like) having appropriate dimension and shape to achieve the desired torque, flexibility, and/or other characteristic. For example, the filament used to form the coil 31 may be round, flat, oval, rectangular, square, triangle, polygonal, and the like, or any suitable shape. The coil 31 can be wrapped in a helical fashion by conventional winding techniques. The pitch of adjacent turns of coil 31 may be tightly wrapped so that each turn touches the succeeding turn, or the pitch may be set such that coil 31 is wrapped in an open fashion. The pitch can be constant throughout the length of the coil 31, or can vary, depending upon the desired characteristics, for example flexibility. For example, in some embodiments, the coil 31 can include a distal portion including a relatively open pitch, and a proximal portion having a relatively more closed pitch, such that the coil is more flexible in the distal portion than in the proximal portion. The reinforcing layer 31 may extend the entire length of the inner tubular member 24, or may extend only along a portion of the length thereof.

The inner tubular member 24 can be constructed using any one or a combination of appropriate methods and/or techniques, for example, extrusion, co-extrusion, interrupted layer co-extrusion (ILC), heat bonding techniques, heat shrink techniques, fusing, winding, disposing, adhesive bonding, mechanical bonding, soldering, welding, molding, casting, or the like, or others. In some embodiments, one or more of the layers and/or structures 30/31/32/34 can be formed separately, and thereafter coupled and/or connected together, while in some embodiments, one or more of the layers and/or structures 30/31/32/34 can be formed together using suitable techniques.

For example, in some embodiments, the layers and/or structures 30/31/32/34 can be formed separately, such as by extrusion, co-extrusion, interrupted layer co-extrusion (ILC), casting, molding, heat shrink techniques, fusing, winding, or the like, and thereafter coupled or connected together using suitable techniques, such as heat shrink techniques, friction fitting, mechanically fitting, chemically bonding, thermally bonding, welding (e.g., resistance, Rf, or laser welding), soldering, brazing, adhesive bonding, crimping, or the use of a connector member or material, or the like, or combinations thereof, to form the inner tubular member 24.

In some other embodiments, one or more of the layers and/or structures of the inner tubular member may be formed together at the same or similar times using suitable techniques, such as extrusion, co-extrusion, interrupted layer co-extrusion (ILC), or the like. In some other embodiments, one or more layers, for example the inner layer 34 and the reinforcing layer 31, can be formed and/or provided separately, and thereafter additional layers, for example layers 32 and 30, can be formed onto, over, or with the layers 31 and 34 by suitable techniques to form the inner tubular member 24.

The inner tubular member 24 may have a uniform stiffness, or may vary in stiffness along its length. For example, a gradual reduction in stiffness from the proximal end to the distal end thereof may be achieved, depending upon the desired characteristics. The gradual reduction in stiffness may be continuous or may be stepped, and may be achieved, for example, by varying the structure, such as the size, thickness, or other physical aspect of one or more of the layers 30/31/32/34, or for example, by varying the materials used in one or more of the layers 30/31/32/34. Such variability in characteristics and materials can be achieved, for example, by using techniques such as ILC, by fusing together separate extruded tubular segments, or in some cases, varying the characteristics and/or even the very presence or absence of certain structures and/or layers.

Referring to FIG. 3, the outer member 26 can also be a generally tubular member including a proximal region 36 having a proximal end 37 and a distal region 38 having a distal end 39. The outer member 26 can be disposed about at least a portion of the inner tubular member 24 at a location along the length of the shaft 12 between proximal end 18 and distal end 22. In the embodiment shown, the outer member 26 is disposed about the inner tubular member 26 along the proximal portion 16 of the shaft 12, but it should be understood that other locations are possible.

The length of the outer member 26 can also vary, depending upon, for example, the length of the shaft 12, the desired characteristics and functions of the catheter 10, and other such parameters. In some embodiments, the outer member 26 has a length that allows it to be disposed over the majority of the length of the inner tubular member 24, and in some embodiments, is disposed about all but up to the distal most 15 cm or less of the inner tubular member 24 and/or all but the proximal most 15 cm or less of the inner tubular member 24. In some embodiments, the length of the outer tubular member 26 can be in the range of about 1-299 centimeters or more, or in some embodiments in the range of about 19 cm-249 cm.

The outer member 26 defines a lumen 40 that can be adapted and/or configured to house or surround a portion of the inner tubular member 24. In some embodiments, the lumen 40 can have an inner diameter that is in the range of about 0.015 to about 0.06 inch in size, and in some embodiments, in the range of about 0.02 to about 0.035 inch in size. In some embodiments, the outer member 26 can have an outer diameter that is in the range of about 0.016 to about 0.07 in size, or in the range of about 0.02 to about 0.04 inch in size. It should be understood however, that these, and other dimensions provided herein, are by way of example only.

In at least some embodiments, the outer tubular member 26 can have an inner diameter that is greater than the outer diameter of the inner tubular member 24. As such, the outer tubular member 26 can be disposed about the inner tubular member 24 (i.e. a portion of the inner tubular member 24 is disposed within the lumen 40 of the outer member) such that a space or gap 42 is defined between at least a portion of the outer surface 25 of the inner tubular member 24 and the inner surface 27 of the outer member 26. In some embodiments, the space or gap 42 can be in the range of about 0.0002 to about 0.004 inch in size, and in some embodiments, in the range of about 0.0005 to about 0.003 inch in size. Again, it should be understood that these dimensions are provided by way of example only.

Typically, relatively large portions of the gap or space 42 remain open or unfilled by any other structure of the catheter 10 along a substantial portion of the length thereof, and in some cases along a substantial portion of the length of the outer member 26. For example, in some embodiments, 50% or more, 75% or more, 90% or more, or 95% or more of the gap or space 42 remains open and/or unfilled by any other structure of the catheter.

In some embodiments, attachment points along the length of the outer member 26 may be used to attach to the inner tubular member 24. As a result, the gap or space 42 may be partially or totally filled at these attachment points, and as such, divided up into what may be considered multiple and/or a plurality of separate gaps or spaces that are unfilled. Additionally, other structures, such as coils, bands, braids, polymer layers, or the like, may fill portions of the gap or space 42. Even so, such multiple the gap or space 42, or the so defined multiple gaps or spaces 42 may still collectively extend along a substantial portion of the length of the outer tubular member 26 and remain overall substantially unfilled over the majority of the length thereof, for example, in percentages of the total length as given above. As such, the outer tubular member 26 can act to reinforce or impart desired properties, such as tortional and lateral rigidity, to the catheter shaft 12, and may allow at least the portion of the inner tubular member 24 surrounded by the gap or space 42 to be separate from, and in some cases bend and/or move laterally within, the lumen 40. Some examples of structure, methods, and techniques of coupling the outer member 26 to the inner tubular member 24 will be discussed in more detail below.

The outer member 26 can be adapted and/or configured to have a desired level of stiffness, torqueability, flexibility, and/or other characteristics. Those of skill in the art and others will recognize that the dimensions, structure, and materials of the outer member 26 are dictated primary by the desired characteristics, and the function of the final catheter 10, and that any of a broad range of the dimensions, structure, and materials can be used.

The desired stiffness, torquability, lateral flexibility, bendability or other such characteristics of the outer member 26 can be imparted or enhanced by the structure of the outer member 26. For example, the outer member 26 may include a thin wall tubular structure, including one or a plurality of apertures 44, such as grooves, cuts, slits, slots, or the like, formed in a portion of, or along the entire length of, the tubular outer member 26. Such structure may be desirable because it may allow outer member 26, or portions thereof, to have a desired level of laterally flexibility as well as have the ability to transmit torque and pushing forces from the proximal region 36 to the distal region 38. The apertures 44 can be formed in essentially any known way. For example, apertures 44 can be formed by methods such as micro-machining, saw-cutting, laser cutting, grinding, milling, casting, molding, chemically etching or treating, or other known methods, and the like. In some such embodiments, the structure of the outer member 26 is formed by cutting and/or removing portions of the tube to form apertures 44.

In some embodiments, the apertures 44 can completely penetrate the outer member 26 such that there is fluid communication between the lumen 40 and the exterior of the outer member 26 through the apertures 44. In some embodiments, the apertures 44 may only partially extend into the structure of the outer member 26, either on the interior or exterior surface thereof. Some other embodiments may include combinations of both complete and partial apertures 44 through the structure of the outer member 26. The shape and size of the apertures 44 can vary, for example, to achieve the desired characteristics. For example, the shape of apertures 44 can vary to include essentially any appropriate shape, such as square, round, rectangular, pill-shaped, oval, polygonal, elongate, irregular, or the like, and may include rounded or squared edges, and can be variable in length and width, and the like.

Additionally, the spacing, arrangement, and/or orientation of the apertures 44, or in some embodiments, associated spines or beams that may be formed, can be varied to achieve the desired characteristics. For example, the number or density of the apertures 44 along the length of the outer member 26 may be constant or may vary, depending upon the desired characteristics. For example, the number or proximity of apertures 44 to one another near one end of the outer member 26 may be high, while the number or proximity of slots to one another near the other end of the outer member 26, may be relatively low and/or non existent, or vice versa. For example, in the embodiment shown in FIGS. 1, 2, and 3, the distal region 38 of the outer member 26 includes a plurality of apertures 44 having a relatively high density relative to the plurality of apertures 44 located in the proximal region 36. As such, the distal region 38 can have a greater degree of lateral flexibility relative to the proximal region 36. The density of the apertures 44 can vary gradually or in a stepwise fashion over the length of the outer tubular member. And as suggested above, certain portions of the outer member 26 may not include any such apertures.

In some embodiments, the distal about 10 to about 50% of the total length of the outer member 26 can include apertures 44 defined therein at a relatively high density, while the proximal about 50 to about 90% of the total length of the outer member 26 include apertures 44 defined therein at a relatively low density, and/or is free of such apertures 44. For example, in some embodiments, the distal region 38 having a length in the range of about 30 to about 70 cm includes apertures 44 defined therein at a relatively high density to provide for relatively greater flexibility, while the remaining length in the proximal region 36 of the outer member 26 include apertures 44 defined therein at a relatively low density, and/or is free of such apertures 44, to provide for relatively greater stiffness. It should be understood however, that these, and other dimensions provided herein, are by way of example embodiments only, and that in other embodiments, the disposition of apertures 44 can vary greatly from the dimensions given, depending upon the desired characteristics and function of the device.

As suggested above, the apertures 44 may be formed such that one or more spines or beams are formed in the tubular outer member 26. Such spines or beams 50 (FIG. 1) could include portions of the tubular member 26 that remain after the apertures 44 are formed in the body of the outer tubular member 26. Such spines or beams 50 may act to maintain a relatively high degree of tortional stiffness, while maintaining a desired level of lateral flexibility. In some embodiments, some adjacent apertures 44 can be formed such that they include portions that overlap with each other about the circumference of the tube. In other embodiments, some adjacent apertures 44 can be disposed such that they do not necessarily overlap with each other, but are disposed in a pattern that provides the desired degree of lateral flexibility. Additionally, the apertures 44 can be arranged along the length of, or about the circumference of, the outer member 26 to achieve desired properties. For example, the apertures 44 can be arranged in a symmetrical pattern, such as being disposed essentially equally on opposite sides about the circumference of the outer member 26, or equally spaced along the length of the outer member, or can be arranged in an increasing or decreasing density pattern, or can be arranged in a non-symmetric or irregular pattern.

Collectively, these figures and this description illustrate that changes in the arrangement, number, and configuration of slots may vary without departing from the scope of the invention. Some additional examples of shaft constructions and/or arrangements of cuts or slots formed in a tubular body are disclosed in U.S. Pat. No. 6,428,489 and in Published U.S. patent application Ser. Nos. 09/746,738 (Pub. No. US 2002/0013540), and 10/400,750 (Pub. No. US-2004-0193140-A1), all of which are incorporated herein by reference. Also, some additional examples of shaft constructions and/or arrangements of cuts or slots formed in a tubular body for use in a medical device are disclosed in U.S. patent application Ser. Nos. 10/375,493, and 10/400,750, which are also incorporated herein by reference.

In addition to, combination with, or as an alternative to the structure of the outer member 26, the materials selected for outer member 26 may also be chosen so that may have the desired characteristics. The outer member 26 may be formed of any materials suitable for use, dependent upon the desired properties of the catheter 10. For example, outer member 26 may be formed of materials having a desired modulus of elasticity—given the structure used. Some examples of suitable materials include metals, metal alloys, polymers, or the like, or combinations or mixtures thereof.

Some examples of suitable metals and metal alloys include stainless steel, such as 304V, 304L, and 316L stainless steel; alloys including nickel-titanium alloy such as linear elastic or superelastic (i.e. pseudoelastic) nitinol; nickel-chromium alloy; nickel-chromium-iron alloy; cobalt alloy; tungsten or tungsten alloys; MP35-N (having a composition of about 35% Ni, 35% Co, 20% Cr, 9.75% Mo, a maximum 1% Fe, a maximum 1% Ti, a maximum 0.25% C, a maximum 0.15% Mn, and a maximum 0.15% Si); hastelloy; monel 400; inconel 625; or the like; or other suitable material, or combinations or alloys thereof. In some embodiments, it is desirable to use metals, or metal alloys that are suitable for metal joining techniques such as welding, soldering, brazing, crimping, friction fitting, adhesive bonding, etc. Some examples of suitable polymeric materials may include, but are not limited to: poly(L-lactide) (PLLA), poly(D,L-lactide) (PLA), polyglycolide (PGA), poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), polyethylene oxide (PEO), polydioxanone (PDS), polycaprolactone (PCL), polyhydroxylbutyrate (PHBT), poly(phosphazene), polyD,L-lactide-co-caprolactone) (PLA/PCL), poly(glycolide-co-caprolactone) (PGA/PCL), polyanhydrides (PAN), poly(ortho esters), poly(phoshate ester), poly(amino acid), poly(hydroxy butyrate), polyacrylate, polyacrylamid, poly(hydroxyethyl methacrylate), polyurethane, polysiloxane and their copolymers, or mixtures or combinations thereof. Some other potentially suitable polymer materials may include those listed above with reference to the inner tubular member 24.

As indicated above, some embodiment may include linear-elastic or super-elastic nitinol in various structures and/or components of the shaft 12 (e.g. outer tubular member 26, inner tubular member 24, etc.). The word nitinol was coined by a group of researchers at the United States Naval Ordinance Laboratory (NOL) who were the first to observe the shape memory behavior of this material. The word nitinol is an acronym including the chemical symbol for nickel (Ni), the chemical symbol for titanium (Ti), and an acronym identifying the Naval Ordinance Laboratory (NOL). In some embodiments, nitinol alloys can include in the range of about 45 to about 60 weight percent nickel, with the remainder being essentially titanium. It should be understood, however, that in other embodiment, the range of weight percent nickel and titanium, and or other trace elements may vary from these ranges. Within the family of commercially available nitinol alloys, are categories designated as “superelastic” (i.e. pseudoelastic) and “linear elastic” which, although similar in chemistry, exhibits distinct and useful mechanical properties.

In some embodiments, a superelastic alloy, for example a superelastic nitinol can be used to achieve desired properties. Such alloys typically display a substantial “superelastic plateau” or “flag region” in its stress/strain curve. Such alloys can be desirable in some embodiments because a suitable superelastic alloy will provide an outer member 26 that is exhibits some enhanced ability, relative to some other non-superelastic materials, of substantially recovering its shape without significant plastic deformation, upon the application and release of stress, for example, during placement of the catheter in the body.

In some other embodiments, a linear elastic alloy, for example a linear elastic nitinol can be used to achieve desired properties. For example, in some embodiments, certain linear elastic nitinol alloys can be generated by the application of cold work, directional stress, and/or heat treatment, such that the material fabricated does not display a substantial “superelastic plateau” or “flag region” in its stress/strain curve. Instead, in such embodiments, as recoverable strain increases, the stress continues to increase in a somewhat linear relationship until plastic deformation begins. In some embodiments, the linear elastic nickel-titanium alloy is an alloy that does not show any martensite/austenite phase changes that are detectable by DSC and DMTA analysis over a large temperature range. For example, in some embodiments, there are no martensite/austenite phase changes detectable by DSC and DMTA analysis in the range of about −60° C. to about 120° C. The mechanical bending properties of such material are therefore generally inert to the effect of temperature over a broad range of temperature. In some particular embodiments, the mechanical properties of the alloy at ambient or room temperature are substantially the same as the mechanical properties at body temperature. In some embodiments, the use of the linear elastic nickel-titanium alloy allows the outer member to exhibit superior “pushability” around tortuous anatomy. One example of a suitable nickel-titanium alloy exhibiting at least some linear elastic properties is FHP-NT alloy commercially available from Furukawa Techno Material Co. of Kanagawa, Japan. Additionally, some examples of suitable nickel-titanium alloy exhibiting at least some linear elastic properties include those disclosed in U.S. Pat. Nos. 5,238,004 and 6,508,803, which are incorporated herein by reference.

In some embodiments, the outer member 26, or other portions of the shaft 12, can be formed of a shape-memory material, for example a shape memory alloy such as a shape memory nitinol. In such embodiments, the shape memory effect can be used in the deployment or use of the catheter, for example in causing the outer member 26, or other portions of the shaft 12, to move from a first insertion configuration to a second use configuration, or, for example, for the outer member 26 to “remember” its desired shape after deformation to another shape.

For example, in some embodiments, the outer member 26 can include or be made of a shape memory alloy that is martensite at room temperature, and has a final austenite transition temperature (A_{f}) somewhere in the temperature range between room temperature and body temperature. For example, in some such embodiments, the shape memory alloy has a final austenite transition temperature in the range of about 25° C. and about 37° C. (e.g. about body temperature). In some such embodiments, it may be desirable that the final austenite transition temperature be at least slightly below body temperature, to ensure final transition at body temperature. This feature allows the outer member 26 to be inserted into the body of a patient in a martensitic state, and assume its preformed, austenitic shape when exposed to the higher body temperature within the anatomy, or at the target site. In this embodiment, deployment of the outer member 26 can be achieved by a shape memory effect—as the material warms, it undergoes a transition from martensite to austenite form, causing transformation of the outer member 26 from the first configuration to the second configuration.

In other example embodiments, the outer member 26 can include or be made of a shape-memory alloy that could have a transition temperature M_{d} (wherein M_{d}=highest temperature to strain-induced martensite) that is in the range of body temperature (e.g. about 37° C.) or greater, below which the alloy retains sufficient stress-induced martensitic property to allow placement of the outer member 26 at or above its final austenite transition temperature (A_{f}). In other words, this allows the catheter, including the outer member 26 in its preformed austenitic state, to be inserted and navigated in the anatomy, where the outer member 26 may be exposed to stress that may promote portions thereof to undergo stress-induced martensitic (SIM) transformation. Thereafter, the outer member 26 may recover its preformed, austenitic shape when released from the stress of navigation, at a temperature that may be substantially above the final austenite transition temperature without significant plastic, or otherwise permanent deformation. Additionally, in some such embodiments, the outer member 26 can be constrained, for example, in a delivery device, such as a guide catheter, in a stress-induced martensitic (SIM) state, and recover its preformed, austenitic shape when released from the constraints of the catheter, at a temperature that may be substantially above the final austenite transition temperature without significant plastic, or otherwise permanent deformation. In these embodiments, the final austenite temperature may be quite low, e.g., 4° C. or lower, or it may be up to room temperature or higher.

In yet other embodiments, the outer member 26 can include or be made of a shape memory alloy that is martensite at body temperature, and has a final austenite transition temperature (A_{f}) somewhere in the temperature range above body temperature. This feature allows the catheter including the outer member 26 to be navigated in a martensitic state, and maintain a martensitic state until exposed to a temperature higher than body temperature. The outer member 26 can then be heated to the necessary temperature above body temperature to make the transformation from martensite to austenite using an external heating means or mechanism. Such mechanisms may include the injection of heated fluid through the catheter, or other device, the use of electrical or other energy to heat the outer member 26, or other such techniques. In some such embodiments, the shape memory alloy has a final austenite transition temperature in the range of about 37° C. to about 45° C. It may be desirable that the final austenite transition temperature be at least slightly above body temperature, to ensure there is not final transition at body temperature. Some examples or Nitinol cylindrical tubes having desired transition temperatures, as noted above, can be prepared according to known methods.

Referring to FIG. 3, the outer tubular member 26 may be connected to the inner tubular member 24 using any of a broad variety of suitable techniques, some examples of which may include adhesive bonding, friction fitting, mechanically fitting, crimping, chemically bonding, thermally bonding, welding (e.g., resistance, Rf, or laser welding), soldering, brazing, or the use of a connector member or material, or the like, or combinations thereof. As discussed above, in at least some embodiments, the outer tubular member 26 can be disposed about the inner tubular member 24 (i.e. a portion of the inner tubular member 24 is disposed within the lumen 40 of the outer member) such that a space or gap 42 is defined between at least a portion of the outer surface 25 of the inner tubular member 24 and the inner surface 27 of the outer member 26.

In FIG. 3, the outer member 26 is attached to the inner tubular member 24 at one or more proximal attachment point 53, one or more distal attachment point 59, and one or more intermediate attachment point 61. In some embodiments, such attachment points can be achieved, for example, using an adhesive material, for example, a cyanoacrylate, or other suitable type of adhesive. In at least some embodiments, only a relatively small portion of the outer member 26 is connected to the inner tubular member 24 at the attachment points. For example, the length of each individual bond joint, especially at the intermediate bond joints, may only be about 5 cm or less, or 3 cm or less, or 1 cm or less, or 0.5 cm or less. In some embodiments, where appropriate, the bonds extend under or within about five or fewer of the apertures 44, or three or even two or fewer of the apertures 44, along the length of the outer tubular member. Some embodiments may include a plurality of intermediate attachment point 61 spaced apart along the length of the shaft 12. In some embodiments, the distance between attachment points along the length of the shaft 12 may be in the range of about 5 cm and about 40 cm, or in the range of about 7 to about 30 cm, and may vary or be constant along the length of the shaft 12. For example, the spacing between attachment points may be closer together near the distal end of the shaft, and may be farther apart near the distal portion of the shaft 12.

As indicated above, the distal portion 20 of the shaft 12 can include a distal tip 28. The distal tip 28 can be a structure, assembly, construction and/or arrangement adapted and/or configured to provide characteristics such as shapability, flexibility, steerability, atraumatic characteristics, or the like, for example, to the distal portion and/or distal end of the shaft 12. A broad variety of distal tip constructions, configurations, and/or structures are generally know for use on medical devices, such as catheters, and may be used. In some embodiments, the distal tip 28 may be disposed at the distal portion 20 of the shaft 12, and may extend distally beyond other portions of the shaft 12.

In some embodiments, the distal tip 28 is simply portions of the shaft 12, and/or components thereof (e.g. the inner and/or outer tubular members 24/26) that include materials and/or structures to provide the desired characteristics. For example, in the embodiment shown in FIG. 3, the distal tip 28 can include and/or extend about the distal portion 20 of the inner tubular member 24. In this regard, the distal tip 28 may include the distal portion 20 of the inner tubular member 24, and may additionally include one or more additional layers and/or structures 52 disposed about the distal portion 20 of the inner tubular member 24. In other embodiments, however, the distal tip 28 may include structure and/or material that may be considered to be separate and distinct from other portions of the shaft, but that is connected to the distal portion of the shaft 12 to form the distal tip.

In FIG. 3, the layer 52 is disposed about the distal portion 20 of the inner tubular member 24. The layers 30, 32, and 34 of the inner tubular member 24 may include distal portions, for example 45 and 49, that include materials having desirable flexibility characteristics, for example, as discussed above. Additionally, the layer 52 may be made of or include any suitable material or structure, and may be disposed by any suitable process, the materials, structures, and processes varying with the particular application and characteristics desired. For example, in some embodiments, the one or more additional layers and/or structures may include a layer of polymer or other such material, or structures such as coils, braids, ribbons, wires, bands, of the like.

In this embodiment, the outer layer 52 may include and/or be made of a polymer material disposed about the distal portion 35 of the inner tubular member 24. For example, the outer layer 52 may include a flexible polymer material having a durometer in the range of about 5 D to about 35 D. Some examples of suitable polymers may include those discussed above with regard to the layers of the inner tubular member 24, with one example being a PEBA material, or the like. As can be appreciated, in some embodiments, the coil layer 31 extends partially into the distal tip 28, but ends and is spaced proximally from the distal end 22. In other embodiments, however, the coil 31, or other such reinforcing structure, or the like, may extend to the distal end 22. Additionally, it should be understood that one or more additional layers and/or constructions may be used in the distal tip 28.

The outer layer 52 may be sized appropriately so as to maintain a generally constant diameter in the transition between the outer member 26 and the outer layer 52, and may include a portion 65 that abuts and/or overlaps the distal end 39 of the outer member 26 to provide a smooth transition. Additionally, as in the embodiment shown, the outer tubular member 26 may include a recessed, or reduced diameter portion at the distal end 39 thereof, and the outer layer 52 may overlap with the recessed portion to provide for a smooth transition. In other embodiments, however, a tapered or step down transition may be provided.

The outer layer 52 can be constructed and/or disposed using any appropriate technique, for example, by extrusion, co-extrusion, interrupted layer co-extrusion (ILC), coating, heat shrink techniques, heat bonding, thermally bonding, casting, molding, fusing one or several segments of an outer layer material end-to-end, adhesive bonding, chemically bonding, crimping, friction fitting, mechanically fitting, or the like, or combinations thereof.

A lubricious, a hydrophilic, a protective, or other type of coating may be applied over portions or the entire shaft 12. Hydrophobic coatings such as fluoropolymers provide a dry lubricity which improves catheter handling and device exchanges. Lubricious coatings can aid in insertion and steerability. Suitable lubricious polymers are well known in the art and may include silicone and the like, hydrophilic polymers such as polyarylene oxides, polyvinylpyrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

It should also be understood that in some embodiments, a degree of MRI compatibility can be imparted into shaft 12. For example, to enhance compatibility with Magnetic Resonance Imaging (MRI) machines, it may be desirable to construct portions of the outer tubular member 26, portions of the inner tubular member 24, or other portions of the shaft 12, in a manner, or use materials that would impart, a degree of MRI compatibility. For example, the lengths of relatively conductive structures within the shaft 12 may be limited to lengths that would not generate undue heat due to resonance waves created in such structures when under the influence of an MRI field generated by an MRI machine. Alternatively, or additionally, portions, or the entire shaft 12 may be made of a material that does not substantially distort the image and create substantial artifacts (artifacts are gaps in the image). Certain ferromagnetic materials, for example, may not be suitable because they may create artifacts in an MRI image. Additionally, all or portions of the shaft 12, may also be made of, impregnated with, plated or clad with, or otherwise include a material and/or structure that the MRI machine can image. Some materials that exhibit these characteristics include, for example, tungsten, Elgiloy, MP35N, nitinol, and the like, and others. Additionally, some structures including or made of such materials, such as marker bands, marker coils, rings, impregnated polymer sections, or the like, may be added to or included in the shaft 12. Those skilled in the art will recognize that these materials can vary widely without departing from the spirit of the invention.

Additionally, all or portions of the shaft 12, or components or layers thereof, may be made of, impregnated with, plated or clad with, or otherwise include a radiopaque material and/or structure to facilitate radiographic visualization. Radiopaque materials are understood to be materials capable of producing a relatively bright image on a fluoroscopy screen or another imaging technique during a medical procedure. This relatively bright image may aid the user of catheter 10 in determining its location. Some examples of radiopaque materials can include, but are not limited to, gold, platinum, palladium, tantalum, tungsten alloy, polymer material loaded with radiopaque filler, and the like.

For example, with reference to FIGS. 1-3, the inner tubular member 24 can include one or more radiopaque marker member 55 disposed in the distal portion 35 between the intermediate and outer layers 32/30, or at other positions and/or locations. Additionally, the outer tubular member 26 can include one or more marker member 57 disposed thereon. In the embodiment shown, the outer tubular member 26 can include one or more recessed portion defined in the outer surface thereof, and one or more marker member 57 is shown disposed therein, for example through crimping. In the embodiment shown, the marker members 55 and 57 are tubular marker bands, but it should be understood that other marker structures and arrangements, such as marker coils, rings, impregnated polymer sections, or the like, may be used, and may be disposed at locations along and/or within the shaft 12. Furthermore, the elongate shaft 12, or portions thereof, may be curved and/or shaped as desired, or be adapted and/or configured to be curved and/or shaped as desired, depending on the particular application.

Now that various example embodiments of shaft configurations have been discussed, the focus now shifts to a discussion of structures of the manifold assembly 14 and the proximal portion 16 of the shaft 12, and methods relating to the connection and/or attachment of the manifold assembly 14 to the shaft 12.

Refer again to FIG. 3—as can be appreciated, the lumen 15 extends through the proximal end 18 of the shaft 12 along longitudinal axis x to form a proximal opening 13 at the proximal end 18 of the shaft 12. The proximal portion 16 of the shaft 12 can include an outwardly flared and/or tapered section and/or segment, for example section 19. In some embodiments, this outwardly flared and/or tapered segment may be adapted and/or configured to mate with and/or connect with a portion of the manifold assembly 14, as will be discussed in more detail below.

In some embodiments, the proximal portion 16 of the shaft 12 can include the section 19 that is flared and/or tapered outwardly such that the inner surface 11 of the shaft 12 within the section 19 extends at an angle away from the longitudinal axis x. For example, the section 19 can be flared and/or tapered such that the inner surface 11 of the shaft 12 within the section 19 extends outwardly at an angle A relative to the longitudinal axis x. The angle A may be in the range of about 1 to about 45 degrees, or in the range of about 5 to about 30 degrees, or in the range of about 10 to about 20 degrees relative to the longitudinal axis x.

Additionally, the proximal portion 16 of the shaft 12 can include a second section 17 disposed distally of, and that may be adjacent to, the first section 19. The second section 17 may be formed such that the inner surface 11 of the shaft 12 within the second section 17 extends in a direction that is generally parallel to the longitudinal axis x, or, in other contemplated embodiments, may extend in a direction that is at an angle different from the longitudinal axis x. In either case, however, the section 19 can be flared and/or tapered outwardly at an angle relative to the second section 17 such that the inner surface 11 of the shaft 12 within the section 19 extends at an angle B relative to the inner surface 11 of the shaft 12 within the second section 17. In some embodiments, angle B may be in the range of about 1 to about 45 degrees, or in the range of about 5 to about 30 degrees, or in the range of about 10 to about 20 degrees. It can be appreciated that in the embodiment shown, the inner surface 11 of the shaft 12 within the second section 17 extends generally parallel to the longitudinal axis x, and as such, the angles A and B will be generally the same for this example. However, it should be understood that this is not required in all embodiments, and that other embodiments may include angles A and B being different.

At the proximal end 18 of the shaft 12, the lumen 15 can have a greater diameter than the diameter of the shaft 15 at a point more distal than the proximal end 18. Additionally, the first section 19 can include a proximal end 21 and a distal end 23, and due to the flaring and/or tapering of the section 19, the inner diameter of the lumen 15 adjacent the proximal end 21 is greater than the inner diameter of the lumen 15 adjacent the distal end 23. Similarly, in at least some embodiments, the inner diameter of the lumen 15 adjacent the proximal opening 13 can be generally larger than the inner diameter of the lumen 15 within the second section 17. The inner surface 11 of the shaft 12 at the proximal end 18 can be disposed and terminate at a point that is further from the longitudinal axis x than the inner surface 11 of the shaft 12 within the second section 17.

In the embodiment shown, the section 19 is flared and/or tapered in a generally linear fashion proximally and outwardly. In other embodiments, however, the flaring and/or tapering may occur in a curvilinear or step-wise fashion, which may or may not be uniform. For example, the angles A and B may be constant, or may vary along the length of the tapered and/or flared section 19. In some embodiments, the flared section 19 may have a length in the range of about 0.1 to about 10 cm, or in the range of about 0.5 to about 5 cm. However, it should be understood that these lengths are given by way of example only, and that it is contemplated that other lengths may be used.

Referring still to FIG. 3, in some embodiments, the thickness of proximal portion 16 of the shaft 12 can be varied and/or enhanced. For example, one or more layers of material can be disposed about the proximal portion 16 of the shaft to achieve a thicker outer diameter. For example, in some embodiments, such additional layers and/or structures may provide the proximal portion 16 of the shaft with an increased diameter, for example, to allow for better mating, connection, and/or bonding with structure in the manifold assembly 14. In that regard, the proximal portion 16 of the shaft 12 can include a first 105, and in some cases a second 107, or more, layers disposed about at least a part of the proximal portion 16 of the shaft 12. These layers 105 and 107 can include any suitable materials, for example those discussed above, and may be disposed and/or created using any suitable technique, for example, those discussed above.

For example, in some embodiments, the layer 105 may include and/or be made of a co-extruded tube including an inner portion of an modified polyolefin adhesive resin, such as ADMIR®, and an outer portion including a flexible polymer material, such as PEBA material. The adhesive resin portion may act as a tie layer between the shaft 12 and the layer 105. Additionally, the outer layer 107 may also include a tube of a flexible polymer material, such as PEBA material. Both the inner and outer tubes can be disposed about the shaft, and attached thereto, for example, using thermal attachment techniques, such as thermal bonding, heat treating and/or bonding, shrink tube bonding, or the like.

As can be appreciated from FIG. 3, in some embodiments, the proximal portion 16 of the shaft 12 may include a region 140 that may include a reduced outer diameter relative to other portions of the shaft 12. For example, in the embodiment shown, only the inner layer 34 of the inner tubular member 24 extends into the proximal region 140, while the outer tubular member 26, and the other layers/structures of the inner tubular member 24 all end distally of the region 140. As can be appreciated, the sections 19 and 17 may extend at least partially within the region 140. In some embodiments, this reduced outer diameter region 140 is provided such that the flared section 19 can be created using certain methodology, some examples of which are discussed below. However, this is not necessary in all embodiments. In any regard, the provision of the one or more layers and/or structures about the proximal portion 16 of the shaft, for example, layers 105 and 107, may help to build up the outer diameter of the proximal portion 16 of the shaft 12, for example, over the region 140. As can be seen, in some embodiments, the layers 105 and 107 may extend over other portions of the shaft as well. For example, layers 105 and 107 are shown extending over the proximal end 37 of the outer tubular member 26.

Referring still to FIG. 3, it can be appreciated that the proximal end 18, including the opening 13 includes and/or defines what can be described as a mouth or funnel region 29. The inside diameter of the mouth and/or funnel region 29 can be larger than the inside diameter of the lumen 15 along other portions of the shaft 12, and as indicated above, can include a tapering change in diameter. Additionally, the outside diameter of the mouth and/or funnel region 29 may be greater than that of the original shaft at that location due to the provision of, for example, diameter enhancing layers 105 and 107.

In at least some embodiments, these features, either alone, or in combination, may provide for certain desirable characteristics. For example, and as will be seen in some example embodiments below, with such a mouth and/or funnel region 29, the proximal end 18 of the shaft 12 may be better able to mate with, receive, and/or extend into corresponding mating and/or attachment structure within a manifold assembly 14 (discussed in more detail below). Such mating engagement may provide for a good centering and/or arrangement of the shaft 12 relative to the manifold assembly 14, and may provide for a good connection area. Additionally, in some cases, the mouth and/or funnel region 29 can aid in providing a smooth transition between the manifold assembly 14 and the catheter shaft 12. For example, in some embodiments, the flaring and/or funneling may allow proximal end 18 of the shaft 13 to act similar to a funnel for directing objects into and/or out of the shaft 12 (i.e., the lumen 15 of shaft 12). For example, this feature may make it easier to provide a smooth transition between the shaft 12 and the manifold 14 potentially allowing for easier insertion and/or retraction of other medical devices and/or fluids and/or material into and out of the shaft 12 through the manifold assembly 14.

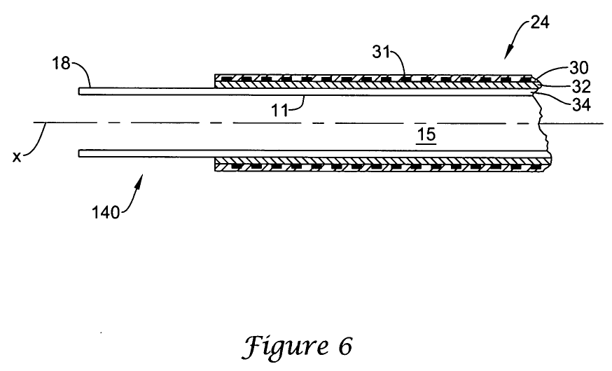

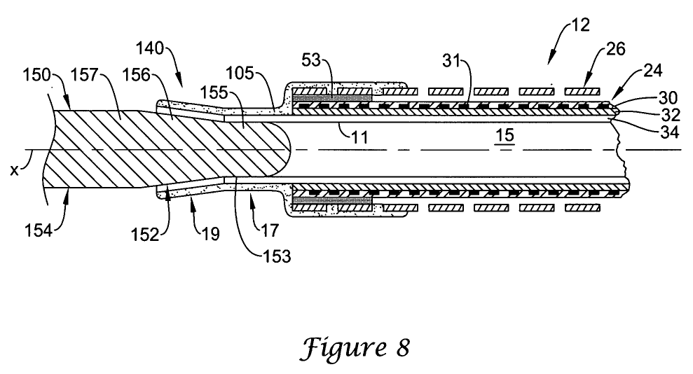

Refer now to FIGS. 6 through 9 for a discussion of some example methods of forming shaft configurations including proximal portions 16 as discussed above. In FIG. 6, the inner tubular member 24, including layers and/or structures 30, 31, 32, and 34 as discussed above, is provided. The inner tubular member 24 can be constructed using methods and materials as discussed above. As can be appreciated, the inner tubular member 24 includes and/or is provided with a proximal region 140 that may include a reduced outer diameter relative to at least some other portions of inner tubular member 24. For example, in the embodiment shown, only the inner layer 34 of the inner tubular member 24 extends into the proximal region 140, while the other layers/structures (30, 31, 32) end distally of the region 140. In some embodiments, the inner tubular member 24 is constructed to have such an arrangement, while in other embodiments, the layers/structures 30, 31, 32 may be initially present at the region 140, and are then removed and/or stripped from the inner tubular member 24 to create the region 140.

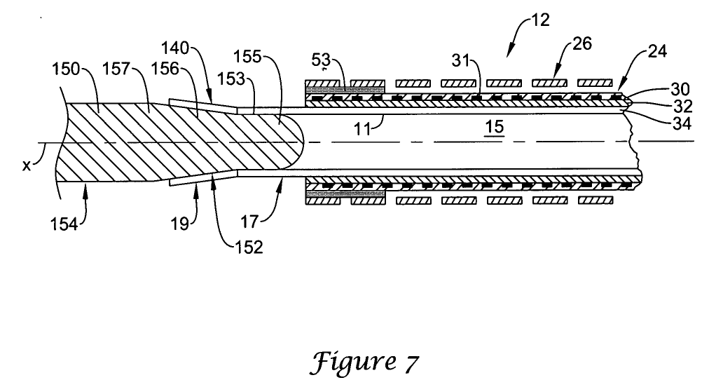

In FIG. 7, a mandrel 150 is shown inserted into the proximal end of the inner tubular member 24 and/or shaft 12 to create the tapered and/or flared section 19. Additionally in FIG. 7, the outer tubular member 26 is shown attached to the inner tubular member 24 to form the shaft 12. As can be appreciated, the attachment of the outer and inner tubular member 24/26 can occur prior to or after the creation of the tapered and/or flared section 19.

The mandrel 150 can have a size and/or shape such that when at least a portion thereof is inserted into the lumen 15, for example at the proximal end of the shaft 12, the mandrel 150 can be used to help create the tapered and/or flared section 19. As such, the mandrel 150, or at least a portion thereof, can include an outer surface that has a shape and/or size that is designed to provide the desired shape and/or size to the tapered and/or flared section 19.

For example, the mandrel 150 may include a distal portion 152 and a proximal portion 154—the distal portion 152 having an outer surface 153 adapted and/or configured (e.g. sized and/or shaped) for insertion into the lumen 15 to aid in creating the tapered and/or flared section 19. The distal section 152 may include, for example, one or more sections that have an outer diameter that is generally larger than the inner diameter of the lumen 15, and as such, when inserted into the lumen 15, can increase the size of the lumen 15 as desired. Additionally, some such sections of the mandrel 150 may be flared and/or tapered to provide such flaring and/or tapering to the lumen 15.

For example, in the embodiment shown, the distal portion 152 of the mandrel 150 may include a first constant diameter section 155, a tapered and/or flared section 156, and a third section 157. The first constant diameter section 155 may include an outside diameter that generally corresponds to the inner diameter of the lumen 15. The tapered and/or flared section 156 may include an outer diameter that generally increases from the first section 155 to the third section 157. The mandrel 150 can be inserted into the lumen 15 such that constant diameter section 155 and at least a portion of the tapered and/or flared section 156 extend into the lumen 15. As can be appreciated, the tapered and/or flared section 156, when inserted into the lumen 15, can be used to deform and/or shape a portion of the lumen 15, thereby creating, for example, a tapered and/or flared section 19. For example, in the embodiment shown, the mandrel 150 is inserted into the proximal region 140 of the inner tubular member 24 and/or shaft 12 to create the tapered and/or flared section 19.

In some embodiments, the mandrel 150 and/or proximal portion of the shaft 12, and/or both, may be heated prior to, during, or after the insertion of the mandrel 150 into the lumen 15. Such heating may aid in allowing the deformation of shaft, as desired, and/or may aid in allowing the shaft to maintain the deformed shape. In other embodiments, however, such heating is not necessary.

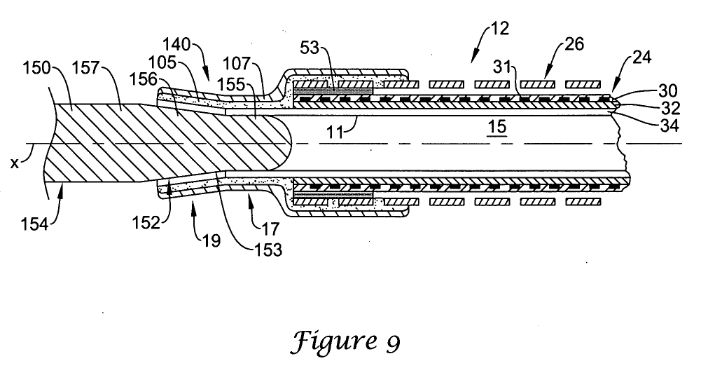

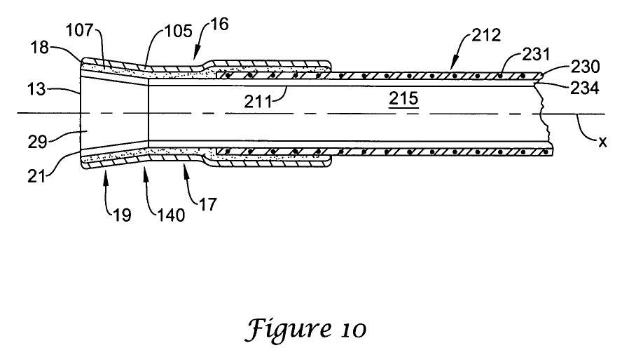

As discussed above, in some embodiments, the thickness of proximal portion 16 of the shaft 12 can be varied and/or enhanced. In that regard, FIGS. 9 and 10 shows the deposition of a first 105 layer and a second layer 107, respectively, about at least a part of the proximal portion 16 of the shaft 12. As indicated above, this layer 105 can include any suitable materials, may be disposed and/or created using any suitable technique. As can be seen, the mandrel 150 may remain disposed within the lumen 15 during the deposition of these layers 105/107, however this is not necessary in all embodiments. It can be appreciated that after the performance of these steps, an elongated shaft having the general structure shown in FIG. 3 may be obtained. It should be understood, however, that a broad variety of other structures and/or configurations may be used. For example, although the embodiments above describe shaft configurations including separate inner and outer tubular members and/or assemblies 24/26, such configurations are not necessary.

For example, refer now to FIG. 10, which shows one example embodiment of an alternative shaft 212 that in some respects is similar to those shown above, but that includes a single tubular assembly rather than two or more tubular assemblies to form the shaft 212—wherein like reference numbers are used to refer to similar structure discussed in the embodiments above. The shaft 212 extends along a central or longitudinal axis x, and includes proximal portion 16 having a proximal end 18, and defines a lumen 215 extending along the longitudinal axis x. The shaft 212 may include a single or multi-layer tubular construction, but in this embodiment includes a multi-layer tubular construction including an inner layer 234, a reinforcing layer 231, and an outer layer 230. The layers 230, 231, and 234 may include materials, structures, and construction similar to layers 30, 31, 32, and 34 discussed in the embodiments above. Additionally, it should be understood that more or fewer layers can be used, with or without one or more reinforcing layers.

As can be appreciated, proximal portion 16 of the shaft 212 can include a first section 19 that is flared and/or tapered outwardly such that the inner surface 211 of the shaft 212 within the first section 19 extends at an angle away from the longitudinal axis x. The first section 19 can be flared and/or tapered as described above with reference to other embodiments.

Additionally, as can be appreciated from FIG. 10, the proximal portion 16 of the shaft 212 may include a region 140 that may include a reduced outer diameter relative to other portions of the shaft 212, and may also include one or more layers and/or structures about the proximal portion 16 of the shaft, for example, layers 105 and 107, as discussed in the embodiments above. As such, it should be understood that at least some of the structures and/or methods discussed in the embodiments above may be used in a broad variety of shaft configurations.

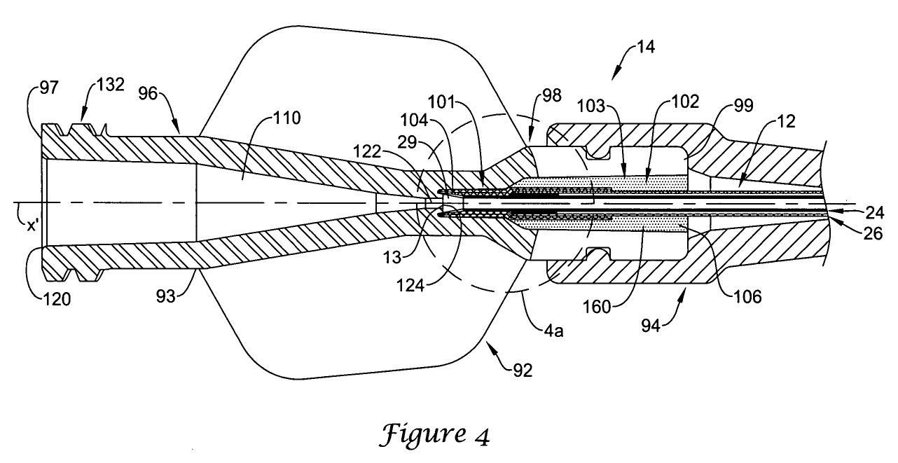

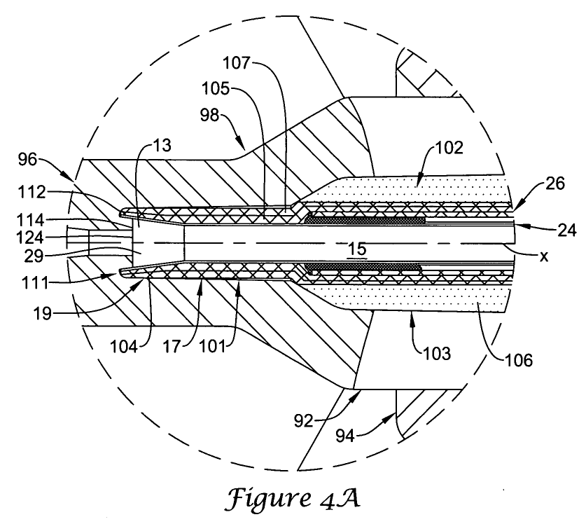

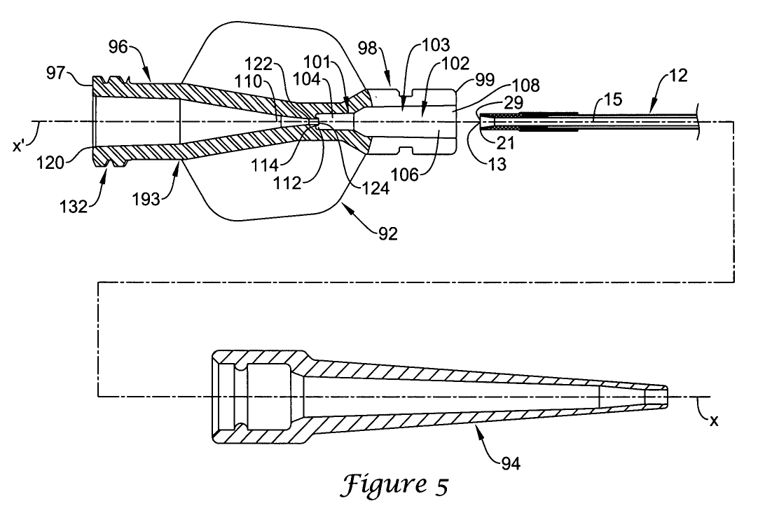

Refer now to FIGS. 4, 4A, and 5, for a discussion of some example embodiments of the manifold assembly 14. The manifold assembly 14 can be made of materials and include structure as generally known in the art, and may include a hub structure and/or portion 92 and a strain relief structure and/or portion 94.

The hub 92 can include structure that can be adapted and/or configured to be connected to and/or mate with the shaft 12. For example, the hub 92 may include structure adapted to receive, and in some cases, mate with, for example, the proximal portion 16 of the shaft 12 or 212 discussed above. The hub 92 may include structure adapted and/or configured to allow for and/or define a pathway for insertion of a medical device, fluid, or other material through the hub 92 and into the lumen 15 of the shaft 12. Additionally, the hub 92 may include structure adapted and/or configured to allow connection of the hub 92, and thus the catheter 10, to other structures and/or devices, such as a Luer fitting, a valve, such as a hemostatic valve, a sealing device, an inflation and/or fluid delivery device, or other fittings, valves, devices, of the like, many of which are well known in the art. For example, in the embodiment shown, threads 132 may be provided on the outer surface of the hub 92 for threadable connection, for example, to Luer fittings.

The strain relief 94 may include structure that may extend about a portion of the hub 93 and a part of the proximal portion 16 of the shaft 12, and allow for desired flexibility characteristics, for example, to provide for a transition in flexibility between the shaft assembly 12 and the remainder of the manifold 14 and/or hub 92. For example, the strain relief 94 can include varying geometry, such as a tapering thickness, grooves, channels, ridges, or other such structure defined, for example, in the surface thereof to provide for the desired flexibility characteristics. In at least some embodiments, the strain relief portion 94 can be more flexible at the distal end than it is at the proximal end thereof. It should be understood that any of a broad variety of structures may be used for the strain relief portion to achieve the desired characteristics. Some examples of strain relief configurations that may be used are disclosed in U.S. Pat. No. 6,273,404, which is incorporated herein by reference. The strain relief portion 94 can be disposed over and connected with the hub 93 and/or the shaft 12 using any of a broad variety of structures and/or methods generally known in the art, for example, by adhesive bonding, friction fitting, mechanically fitting, chemically bonding, thermally bonding, heat shrink materials, molding, casting, welding (e.g., resistance or laser welding), soldering, brazing, the use of an outer sleeve or polymer layer to bond or connect the components, or the like, or combinations thereof.

In some embodiments, the hub 92 can include a hub body 93 including a proximal portion 96 having a proximal end 97, and a distal portion 98 having a distal end 99. The proximal portion 96 of the body 93 defines a lumen 110 extending therein, the lumen 110 including a proximal end or port 120 and a distal end 122. The distal portion 98 defines a cavity and/or pocket 102 extending therein, the pocket 102 including a proximal portion 101 including a proximal end 104 and a distal portion 103 including a distal end 106. In some embodiments, the distal portion 103 of the pocket 102 may include a larger inner diameter than the proximal portion 101. The lumen 110 and the pocket 102 may generally extend along a common axis x′, and are in fluid communication through an opening 124 defined at the junction between the lumen 110 and the pocket 102.

The pocket 102, and/or the structure of the hub body 93 that defines the pocket 102, can be adapted and/or configured to receive, and in some cases, mate with, at least a proximal portion of a catheter shaft, for example, the proximal portion 16 of the shaft 12 or 212 discussed above. For example, the pocket 102 can have an inner diameter that is adapted to receive the proximal end 18 and at least a part of the proximal portion 16 of the shaft 12. Furthermore, the hub body 93 can define locating, attachment, and/or connection structure that is adapted and/or configured to mate with and/or receive the proximal end 18 of the shaft 12.

For example, referring to FIG. 4A, one or more protrusions 114 and/or one or more recessed portions 112 may be defined in the hub body 93 about the opening 124. The protrusion 114 and/or recessed portion 112 may extend generally annularly about the opening 124—the protrusion 114 extending distally out into the pocket 102, and the recessed portion extending proximally away from the pocket 102. For example, the opening 124 can be surrounded by and/or to some extent defined by the protrusion 114. For another example, the protrusion 114 can be surrounded by and/or to some extent defined by the recessed portion 112. As such, either the protrusion 114 or recessed portion 112, or both, can define a locating structure (i.e. “locating dome”) or mechanism such that when the proximal portion 16 of the shaft 12 is advanced into the pocket 102, the proximal end 18 of the shaft 12, including the mouth and/or funnel region 29, mates therewith. For example, the flared and/or tapered proximal end 18 of the shaft 12 can mate with and extend into the recessed portion 112. Furthermore, the mouth and/or funnel region 29 of the shaft 12 can receive and mate with at least a portion of the protrusion 114, for example, such that a portion of the protrusion 114 extends into the lumen 15 of the shaft 12.

Furthermore, in some embodiments, the proximal portion 101 of the pocket 102 can include an inner diameter that generally corresponds to the outer diameter of the proximal portion 16 of the shaft 12 adjacent the proximal end 18. Stated differently, the proximal portion 16 of the shaft 12 may include an outer diameter and/or thickness that generally corresponds to the inner diameter of the proximal portion 101 of the pocket 102. In some embodiments, this can be achieved, for example, by providing outer layers, for example layers 105/107, on the proximal portion of the shaft 12 to achieve the desired diameter/thickness. As such, it can be appreciated that the relative sizing of the proximal portion 101 of the pocket 102 and the proximal portion 16 of the shaft 12 may aid in mating the shaft 12 to the manifold 14 in an appropriate manner.

As can be appreciated, and as discussed at least somewhat above with regard to the mouth and/or funnel region 29 of the shaft 12, such mating structure on the hub body 93 may aid in aligning the shaft 12 with the manifold assembly 14, may provide for a good connection area, and may aid in providing a smooth transition between the manifold assembly 14 and the catheter shaft 12. In at least some embodiments, the combination of the mouth and/or funnel region 29 of the shaft 12, as discussed above, with mating structure on the hub body 93 may provide significant advantages in achieving alignment and/or attachment between the manifold assembly 14 and the shaft 12.

Once appropriately positioned, the hub 92 may be secured to the shaft 12 using any suitable technique, for example, by adhesive bonding, friction fitting, mechanically fitting, chemically bonding, thermally bonding, heat shrink materials, molding, casting, welding (e.g., resistance or laser welding), soldering, brazing, the use of an outer sleeve or polymer layer to bond or connect the components, or the like, or combinations thereof. In some embodiments, portions, or all of any remaining space within the pocket 102 that is not filled with the shaft 12 may be filled with adhesive, for example, a U.V. curable adhesive 160, and thereafter cured or allowed to cure, for example using U.V. energy, to attach the hub 93 to the shaft 12. In some embodiments, a two step process is used, wherein an initial amount of adhesive is applied to the proximal portion of the shaft 12, and the shaft is advanced into mating engagement with the hub 92, and the adhesive is allowed to cure to create a bond between the shaft 12 and the hub 92, for example, in and/or adjacent the proximal portion 101 of the pocket 102. Thereafter, a second amount of adhesive can be inserted and/or otherwise applied into the pocket 102, for example into the distal portion of the pocket, and is allowed to cure.

Once connected, the hub lumen 108 defines pathway between the proximal end or port 120 of the hub and the lumen 15 of the shaft 12 through the hub body 93. The pathway may, for example, allow for the insertion of a medical device, fluid, or material through the hub and into the lumen 15 of the shaft. As can be appreciated, once the hub is attached to the shaft, the longitudinal axis x′ of the hub may generally coincide with the longitudinal axis x of the shaft 12, but this is not necessary in all embodiments.

The manifold assembly 14, including the hub 92 and strain relief 94, may be made using any of a broad variety of suitable methods and using any of a broad variety of suitable materials. For example, the manifold assembly 14, or portions thereof, can be made using molding, casting, shaping, or other forming techniques generally known. For example, the manifold assembly 14, or portions thereof, can be made using injection molding and/or insertion molding techniques. In some other embodiments, rather than being prefabricated and thereafter attached to the proximal portion of the shaft 12, it is contemplated that manifold assembly 14 may be initially made onto the proximal portion of the shaft 12, for example, by molding, casting, shaping, or otherwise forming the hub onto the proximal end 16 of the shaft 12.

Any of a wide variety of materials may be used for the manifold assembly 14, or the components thereof. For example, any of a wide variety of polymers may be used. The hub portion and the strain relief portion may be made out of the same or different materials. Some examples of polymer material may include those already discussed above with regard to other components of the catheter 10, and may also include polycarbonate, polyamide, nylon, polyether block amide (PEBA), thermoplastic vulcanizates, or mixtures, combinations and/or copolymers thereof, or any other suitable material. In some embodiments, the hub portion 92 can be made of a nylon material, and the strain relief 94 can be made of a thermoplastic vulcanizate, such as Santoprene which is commercially available from Advanced Elastomer Systems, LP.

The present invention should not be considered limited to the particular examples described above, but rather should be understood to cover all aspects of the invention as fairly set out in the attached claims. Various modifications, equivalent processes, as well as numerous structures to which the present invention may be applicable will be readily apparent to those of skill in the art to which the present invention is directed upon review of the instant specification. It should be understood that this disclosure is, in many respects, only illustrative. Changes may be made in details, particularly in matters of shape, size, and arrangement of steps without exceeding the scope of the invention. The scope of the invention is, of course, defined in the language in which the appended claims are expressed.

What is claimed is:

1. A medical catheter comprising:
an elongated shaft defining a lumen extending along a longitudinal axis, the shaft including a proximal portion having a proximal end and a distal portion having a distal end;
the proximal portion of the shaft including a flared section disposed adjacent the proximal end, the flared section being flared outwardly at an angle relative to the longitudinal axis of the shaft.

2. The medical catheter of claim 1, wherein the shaft defines an inner surface that defines the lumen, and the flared section is flared outwardly such that the inner surface of the shaft within the flared section extends proximally at an angle away from the longitudinal axis.

3. The medical catheter of claim 1, wherein the shaft defines an inner surface that defines the lumen, and wherein the proximal portion of the shaft includes a second section disposed distally of the flared section, wherein the inner surface of the shaft within the second section extends in a first direction relative to the longitudinal axis, and the inner surface of the shaft within the flared section extends outwardly at an angle relative to the first direction.

4. The medical catheter of claim 3, wherein the first direction extends parallel with the longitudinal axis of the shaft.

5. The medical catheter of claim 1, wherein the lumen has a greater diameter at the proximal end of the shaft than the diameter of the lumen at a point more distal along the shaft.

6. The medical catheter of claim 1, wherein the flared section includes a proximal end and a distal end, and due to the flaring of the flared section, the inner diameter of the lumen adjacent the proximal end of the flared section is greater than the inner diameter of the lumen adjacent the distal end of the flared section.

7. The medical catheter of claim 1, wherein the flared section is flared or tapered in a generally linear fashion proximally and outwardly.

8. The medical catheter of claim 1, wherein the flared section is flared in a curvilinear or step-wise fashion.

9. The catheter of claim 1, wherein the flared section defines a mouth having an inside diameter that is greater than an inside diameter of the lumen along the remainder of the shaft.

10. The medical catheter of claim 1, further comprising:
a hub connected to the proximal end of the shaft, the hub including a hub lumen in fluid communication with the lumen of the shaft, wherein the hub includes one or more protrusion extending there from that are disposed at least partially within the flared section of the shaft.

11. The medical device of claim 10, wherein the hub defines a distal opening in fluid communication with the hub lumen, and the one or more protrusions extend annularly about the distal opening.

12. The medical device of claim 10, wherein the hub includes one or more recessed portion defined therein, and the flared section of the shaft is disposed at least partially within the recessed portion.

13. The medical device of claim 12, wherein the hub defines a distal opening in fluid communication with the hub lumen, and the one or more recessed portions extend annularly about the distal opening.

14. The medical catheter of claim 10, wherein the hub is connected to the proximal end of the shaft using U.V. curable adhesive.

15. A hub for use in medical device applications, the hub comprising:
a body including a proximal portion having a proximal end and a distal portion having a distal end;
a lumen defined in the proximal portion, and a pocket defined in the distal portion, wherein the lumen and the pocket and are in fluid communication through an opening defined at a junction between the lumen and the pocket;
one or more protrusion defined in the body about the opening and extending into the pocket.

16. A hub for use in medical device applications, the hub comprising:
a body including a proximal portion having a proximal end and a distal portion having a distal end;
a lumen defined in the proximal portion, and a pocket defined in the distal portion, wherein the lumen and the pocket and are in fluid communication through an opening defined at a junction between the lumen and the pocket;
one or more recessed portion defined in the body about the opening.

17. A method of making a medical catheter, the method comprising:
providing an elongated shaft defining a lumen extending along a longitudinal axis, the shaft including a proximal portion having a proximal end and a distal portion having a distal end, the proximal portion of the shaft including a flared section disposed adjacent the proximal end, the flared section being flared outwardly at an angle relative to the longitudinal axis of the shaft;
providing a hub including a hub body defining a lumen having an opening, wherein the hub includes one or more protrusion defined in the body about the opening;
disposing the flared section of the shaft about at least a portion of the protrusions such that the lumen of the shaft and the lumen of the hub are in fluid communication; and
attaching the hub to the shaft.

18. The method of claim 17, wherein the hub is attached to the shaft using U.V. curable adhesive.

19. A method of making a medical catheter, the method comprising:
providing an elongated shaft defining a lumen extending along a longitudinal axis, the shaft including a proximal portion having a proximal end and a distal portion having a distal end, the proximal portion of the shaft including a flared section disposed adjacent the proximal end, the flared section being flared outwardly at an angle relative to the longitudinal axis of the shaft;
providing a hub including a hub body defining a lumen having an opening, wherein the hub includes one or more recessed portions defined in the body about the opening;
disposing the flared section of the shaft within at least a portion of the recessed portion such that the lumen of the shaft and the lumen of the hub are in fluid communication; and
attaching the hub to the shaft.

20. A medical catheter comprising:
an elongated shaft defining a lumen extending along a longitudinal axis, the shaft including a proximal portion having a proximal end and a distal portion having a distal end, the proximal portion of the shaft including means for mating with a hub.

21. A hub for use with a medical catheter, the hub comprising:
a body including a proximal portion having a proximal end and a distal portion having a distal end, and a lumen defined by the body having a distal opening;
means for mating with an elongated shaft of a catheter.

22. A medical catheter comprising:
an elongated inner tubular member including a proximal portion and a distal portion and defining an outer surface and a lumen, the inner tubular member including a first layer of polymer material and a reinforcing member; and
an elongated outer tubular member having an outer surface and an inner surface, and including a plurality of apertures defined in the outer surface thereof, the outer member being disposed about the inner tubular member such that at least a portion of the outer surface of the inner tubular member is spaced from the inner surface of the outer member.

23. The medical catheter of claim 22, wherein the inner tubular member further includes one or more layers of polymer material, and wherein the reinforcing member is disposed between two layers of polymer material.

24. The medical catheter of claim 22, wherein the outer tubular member is disposed about the proximal portion of the inner tubular member such that the distal portion of the inner tubular member is free of the reinforcing member.

25. A medical catheter comprising:
an elongated inner tubular member including a proximal portion and a distal portion and defining an outer surface and a lumen;
a elongated outer tubular member having an proximal end and a distal end, and defining an outer surface and an inner surface, and including a plurality of apertures defined in the outer surface, the distal end of the outer tubular member including a reduced diameter portion that has an outer diameter that is smaller than the outer diameter along at least one other portion of the outer tubular member adjacent thereto;
the outer tubular member being disposed about the inner tubular member such that the distal portion of the inner tubular member is free of the outer tubular member and such that a space is formed between the outer surface of the inner tubular member and the inner surface of the outer tubular member along a portion of the proximal portion; and
a distal tip structure coupled to the distal portion of the inner tubular member and the distal end of the outer tubular member, wherein at least a portion of the distal tip structure overlaps the reduced diameter portion of the outer tubular member.

26. The medical catheter of claim 22, wherein the tip structure includes one or more layers of material disposed on the distal portion of the inner tubular member.
